(19) United States
(12) Patent Application Publication
(10) Pub. No.: US 2025/0177321 A1
(43) Pub. Date: Jun. 5, 2025

(54) USE OF CANNABINOIDS IN THE TREATMENT OF EPILEPSY

(71) Applicant: GW Research Limited, Cambridge (GB)

(72) Inventors: Geoffrey GUY, Cambridge (GB); Volker Knappertz, Cambridge (GB)

(21) Appl. No.: 18/912,442

(22) Filed: Oct. 10, 2024

Related U.S. Application Data
(63) Continuation of application No. 17/426,442, filed on Jul. 28, 2021, now abandoned.

(30) Foreign Application Priority Data
Feb. 22, 2019 (GB) 1902427.2

(51) Int. Cl. A61K 31/05; A61K 31/352; A61K 45/06; A61K 47/10; A61K 47/26; A61K 47/44; A61K 47/46; A61P 25/08

(52) U.S. Cl. CPC A61K 31/05; A61K 31/352; A61K 45/06; A61K 47/10; A61K 47/26; A61K 47/44; A61K 47/46; A61P 25/08

(57) ABSTRACT

The present invention relates to the use of cannabidiol (CBD) in the treatment of epilepsy which results from mutation of the KCNT1 gene. The CBD used is in the form of a highly purified extract of Cannabis such that the CBD is present at greater than 98% of the total extract (w/w) and the other components of the extract are characterised. In particular the cannabinoid tetrahydrocannabinol (THC) is present in an amount of from 0.02 to 0.1% (w/w). In an alternative embodiment the CBD may be in a synthetic form. In use the CBD may also be used concomitantly with one or more other anti-epileptic drugs (AED). The CBD may be formulated for administration separately, sequentially or simultaneously with one or more AED or the combination may be provided in a single dosage form. Where the CBD is formulated for administration separately, sequentially or simultaneously it may be provided as a kit or together with instructions to administer the one or more components in the manner indicated. It may also be used as the sole medication, i.e. as a monotherapy.

### FIELD OF THE INVENTION

The present invention relates to the use of cannabidiol (CBD) in the treatment of epilepsy which results from mutation of the KCNT1 gene.

The CBD used is in the form of a highly purified extract of Cannabis such that the CBD is present at greater than 98% of the total extract (w/w) and the other components of the extract are characterised. In particular the cannabinoid tetrahydrocannabinol (THC) is present in an amount of from 0.02 to 0.1% (w/w). In an alternative embodiment the CBD may be in a synthetic form.

In use the CBD may also be used concomitantly with one or more other anti-epileptic drugs (AED). The CBD may be formulated for administration separately, sequentially or simultaneously with one or more AED or the combination may be provided in a single dosage form. Where the CBD is formulated for administration separately, sequentially or simultaneously it may be provided as a kit or together with instructions to administer the one or more components in the manner indicated. It may also be used as the sole medication, i.e. as a monotherapy.

### BACKGROUND TO THE INVENTION

Epilepsy occurs in approximately 1% of the population worldwide, (Thurman et al., 2011) of which 70% are able to adequately control their symptoms with the available existing anti-epileptic drugs (AED). However, 30% of this patient group, (Eadie et al., 2012), are unable to obtain seizure freedom using the AED that are available and as such are termed as suffering from intractable or “treatment-resistant epilepsy” (TRE).

Intractable or treatment-resistant epilepsy was defined in 2009 by the International League Against Epilepsy (ILAE) as “failure of adequate trials of two tolerated and appropriately chosen and used AED schedules (whether as monotherapies or in combination) to achieve sustained seizure freedom” (Kwan et al., 2009).

Individuals who develop epilepsy during the first few years of life are often difficult to treat and as such are often termed treatment-resistant. Children who undergo frequent seizures in childhood are often left with neurological damage which can cause cognitive, behavioral and motor delays.

Childhood epilepsy is a relatively common neurological disorder in children and young adults with a prevalence of approximately 700 per 100,000. This is twice the number of epileptic adults per population.

When a child or young adult presents with a seizure, investigations are normally undertaken in order to investigate the cause. Childhood epilepsy can be caused by many different syndromes and genetic mutations and as such diagnosis for these children may take some time.

The main symptom of epilepsy is repeated seizures. In order to determine the type of epilepsy or the epileptic syndrome that a patient is suffering from, an investigation into the type of seizures that the patient is experiencing is undertaken. Clinical observations and electroencephalography (EEG) tests are conducted and the type(s) of seizures are classified according to the ILAE classification described below.

The International classification of seizure types proposed by the ILAE was adopted in 1981 and a revised proposal was published by the ILAE in 2010 and has not yet superseded the 1981 classification. In addition, the term “simple partial seizure” has been replaced by the term “focal seizure where awareness/responsiveness is not impaired” and the term “complex partial seizure” has been replaced by the term “focal seizure where awareness/consciousness is impaired”.

Generalised seizures, where the seizure arises within and rapidly engages bilaterally distributed networks, can be split into six subtypes: Tonic-Clonic (grand mal) seizures; Absence (petit mal) Seizures; Clonic Seizures; Tonic Seizures; Atonic Seizures and Myoclonic Seizures.

Focal (partial) seizures where the seizure originates within networks limited to only one hemisphere, are also split into sub-categories. Here the seizure is characterized according to one or more features of the seizure, including aura, motor, autonomic and awareness/responsiveness. Where a seizure begins as a localized seizure and rapidly evolves to be distributed within bilateral networks this seizure is known as a Bilateral convulsive seizure, which is the proposed terminology to replace Secondary Generalized Seizures (generalized seizures that have evolved from focal seizures and no longer remain localized).

Focal seizures where the subject's awareness/responsiveness is altered are referred to as focal seizures with impairment and focal seizures where the awareness or responsiveness of the subject is not impaired are referred to as focal seizures without impairment.

Epileptic syndromes often present with many different types of seizure and identifying the types of seizure that a patient is suffering from is important as many of the standard AED's are targeted to treat or are only effective against a given seizure type/sub-type.

Around 1 in 200 children are diagnosed with a genetic epilepsy each year. Co-existing conditions or symptoms also commonly occur in this group including language problems, cognitive problems and headaches.

One such gene is known as KCNT1 or potassium channel subfamily T, member 1. This gene encodes the K_{Ca}4.1 protein which is a member of the calcium-activated potassium channel protein family. Mutation in the KCNT1 gene may result in a diagnosis of Early Infantile Epileptic Encephalopathy (Ohtahara syndrome) or Epilepsy of Infancy with Migrating Focal Seizures (EIMFS).

Epilepsy of Infancy with Migrating Focal Seizures (EIMFS) was first described in 1995 and is very rare, occurring in between 1 in 200,000 and 1 in 400,000 people. Seizures are often experienced in the first few weeks of life and often increase in frequency where seizures may be experienced up to 100 times each day. Babies usually make very little developmental progress.

Sadly, the outlook for EIMFS is very poor due to the seizures being very difficult to control for more than a few days or weeks at a time. Children with this syndrome often die in childhood from complications of the disorder and those that do survive generally have severe neurological and developmental disorders.

Seizure types in EIFMS are initial sporadic focal motor seizures which evolve within weeks to months into near-continuous seizure clusters and developmental deterioration. Seizures are typically pharmacoresistant, treatments reported with potential benefit in various combinations include bromides, stiripentol and clonazepam, levetiracetam, rufinamide, ketogenic diet and quinidine.

Over the past forty years there have been a number of animal studies on the use of the non-psychoactive cannabinoid cannabidiol (CBD) to treat seizures. For example, Consroe et al., (1982) determined that CBD was able to prevent seizures in mice after administration of pro-convulsant drugs or an electric current.

Studies in epileptic adults have also occurred in the past forty years with CBD. Cunha et al. reported that administration of CBD to eight adult patients with secondary generalized epilepsy resulted in a marked reduction of seizures in 4 of the patients (Cunha et al., 1980).

A study in 1978 provided 200 mg/day of pure CBD to four adult patients, two of the four patients became seizure free, whereas in the remainder seizure frequency was unchanged (Mechoulam and Carlini, 1978).

In contrast to the studies described above, an open label study reported that 200 mg/day of pure CBD was ineffective in controlling seizures in twelve institutionalized adult patients (Ames and Cridland, 1986).

Based on the fact that chronologically the last study to look at the effectiveness of CBD in patients with epilepsy proved that CBD was unable to control seizures, there would be no expectation that CBD might be useful as an anti-convulsant agent.

In the past forty years of research there have been over thirty drugs approved for the treatment of epilepsy none of which are cannabinoids. Indeed, there appears to have been a prejudice against cannabinoids, possibly due to the scheduled nature of these compounds and/or the fact that THC, which is a known psychoactive, has been ascribed as a pro-convulsant (Consroe et al., 1977).

The patent application GB 2,487,712 describes the use of CBD with anti-epileptic drugs and WO 2015/193667 describes the use of CBD in the treatment of treatment resistant epilepsy, in particular patients with FIRES are shown to benefit particularly from the treatment.

A paper published in 2013 suggested that cannabidiol-enriched Cannabis may be efficacious in the treatment of epilepsy. Porter and Jacobson report on a parent survey conducted via a Facebook group which explored the use of Cannabis which was enriched with CBD in children with treatment-resistant epilepsy. It was found that sixteen of the 19 parents surveyed reported an improvement in their child's epilepsy. The children surveyed for this paper were all taking Cannabis that was purported to contain CBD in a high concentration although the amount of CBD present and the other constituents including THC were not known for many of the cases. Indeed, whilst CBD levels ranged from 0.5 to 28.6 mg/kg/day (in those extracts tested), THC levels as high as 0.8 mg/kg/day were reported.

A paper by Press et al. (2015) describes a review of 75 children and adolescents provided with oral Cannabis extract. The responder rate for patients with Lennox-Gastaut syndrome was very high at 88.9%, whereas the rate for other childhood epilepsy syndromes such as Doose syndrome and Dravet syndrome were much lower or showed no improvement at all.

Providing children with TRE with a Cannabis extract that comprises THC, which has been described as a pro-convulsant (Consroe et al., 1977), at a potentially psychoactive dose of 0.8 mg/kg/day, is a concern.

Highly purified CBD has been approved in the U.S. as Epidiolex® (Greenwich Biosciences, Inc) for seizures associated with Lennox-Gastaut syndrome (LGS) or Dravet syndrome (DS) in patients ≥2 years of age. To date there have been no trials or studies of CBD in children and young adults with epilepsy associated with KCNT1 mutation.

The applicant has shown that the administration of a specific composition of CBD has a significant impact on the treatment of a child with a KCNT1 mutation associated EIFMS.

The CBD used is in the form of a highly purified extract of Cannabis such that the CBD is present at greater than 98% of the total extract (w/w) and the other components of the extract are characterised. In particular the cannabinoid tetrahydrocannabinol (THC) is present in an amount of from 0.02 to 0.1% (w/w).

### BRIEF SUMMARY OF THE DISCLOSURE

In accordance with a first aspect of the present invention there is provided Cannabidiol (CBD) for use in the treatment of epilepsy associated with KCNT1 mutation.

In a further embodiment the CBD is used in the treatment of non-seizure symptoms in epilepsy associated with KCNT1 mutation.

Preferably the epilepsy associated with KCNT1 mutation is Epilepsy of Infancy with Migrating Focal Seizures (EIMFS).

Preferably the epilepsy is a treatment resistant epilepsy (TRE).

In a further embodiment the CBD is for use in combination with one or more concomitant anti-epileptic drugs (AED).

In a further embodiment the CBD is present as a highly purified extract of Cannabis which comprises at least 98% (w/w) CBD. Preferably the extract comprises up to 0.1% THC. More preferably the extract comprises between 0.2 and 0.1% (w/w). More preferably the extract further comprises up to 1.0% (w/w) CBDV.

In an alternative embodiment the CBD is present as a synthetic compound.

Preferably the dose of CBD is greater than 5 mg/kg/day. Thus, for a 15 kg patient a dose of greater than 75 mg of CBD per day would be provided. Doses greater than 5 mg/kg/day such as greater than 10/mg/kg/day, greater than 15 mg/kg/day, greater than 20 mg/kg/day and greater than 25 mg/kg/day are also envisaged to be effective.

Preferably the dose of CBD is between 5 and 50 mg/kg/day.

In accordance with a second aspect of the present invention there is provided a method of treating epilepsy associated with KCNT1 mutation comprising administering cannabidiol (CBD) to a subject.

Preferably the subject is a human, more preferably a child or young adult.

### Definitions

Definitions of some of the terms used to describe the invention are detailed below:

The cannabinoids described in the present application are listed below along with their standard abbreviations.

Cannabinoids and their Abbreviations

| | | |
|---|---|---|
| CBD | Cannabidiol | 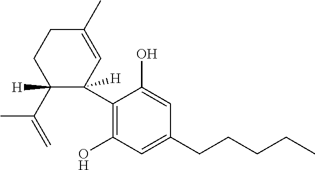 [SMILES: C=C(C)[C@@H]1CCC(C)=C[C@H]1c1c(O)cc(CCCCC)cc1O] |
| THC | Tetrahydrocannabinol | 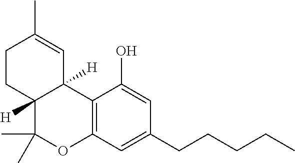 [SMILES: CCCCCc1cc(O)c2c(c1)OC(C)(C)[C@@H]1CCC(C)=C[C@@H]21] |
| CBDV | Cannabidivarin | 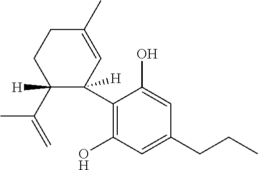 [SMILES: C=C(C)[C@@H]1CCC(C)=C[C@H]1c1c(O)cc(CCC)cc1O] |
| CBD-C4 | Cannabidiol-C4 | 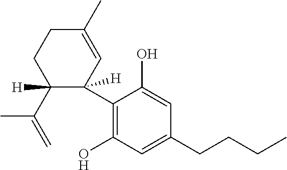 [SMILES: C=C(C)[C@@H]1CCC(C)=C[C@H]1c1c(O)cc(CCCC)cc1O] |
| CBD-C4 | Cannabidiol-C1 | 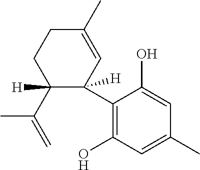 [SMILES: C=C(C)[C@@H]1CCC(C)=C[C@H]1c1c(O)cc(C)cc1O] |

The table above is not exhaustive and merely details the cannabinoids which are identified in the present application for reference. So far over 60 different cannabinoids have been identified and these cannabinoids can be split into different groups as follows: Phytocannabinoids; Endocannabinoids and Synthetic cannabinoids (which may be novel cannabinoids or synthetically produced phytocannabinoids or endocannabinoids).

“Phytocannabinoids” are cannabinoids that originate from nature and can be found in the Cannabis plant. The phytocannabinoids can be isolated from plants to produce a highly purified extract or can be reproduced synthetically.

“Highly purified cannabinoid extracts” are defined as cannabinoids that have been extracted from the Cannabis plant and purified to the extent that other cannabinoids and non-cannabinoid components that are co-extracted with the cannabinoids have been substantially removed, such that the highly purified cannabinoid is greater than or equal to 98% (w/w) pure.

“Synthetic cannabinoids” are compounds that have a cannabinoid or cannabinoid-like structure and are manufactured using chemical means rather than by the plant.

Phytocannabinoids can be obtained as either the neutral (decarboxylated form) or the carboxylic acid form depending on the method used to extract the cannabinoids. For example, it is known that heating the carboxylic acid form will cause most of the carboxylic acid form to decarboxylate into the neutral form.

“Treatment-resistant epilepsy” (TRE) “refractory epilepsy” or “intractable epilepsy” is defined as per the ILAE guidance of 2009 as epilepsy that is not adequately controlled by trials of one or more AED.

“Childhood epilepsy” refers to the many different syndromes and genetic mutations that can occur to cause epilepsy in childhood. Examples of some of these are as follows: Dravet Syndrome; Myoclonic-Absence Epilepsy; Lennox-Gastaut syndrome; Generalized Epilepsy of unknown origin; CDKL5 mutation; Aicardi syndrome; bilateral polymicrogyria; Dup15q; SNAP25; and febrile infection related epilepsy syndrome (FIRES); benign rolandic epilepsy; juvenile myoclonic epilepsy; Sturge Weber Syndrome (SWS); infantile spasm (West syndrome); Landau-Kleffner syndrome and Epilepsy of Infancy with Migrating Focal Seizures (EIMFS). The list above is non-exhaustive as many different childhood epilepsies exist.

Epilepsy of Infancy with Migrating Focal Seizures (EIMFS) is a rare childhood epilepsy. The diagnosis of the syndrome is made by clinical and EEG criteria and corroborated by genetic testing whereby a mutation in the KCNT1 gene is likely to occur.

“Focal Seizures” are defined as seizures which originate within networks limited to only one hemisphere. What happens during the seizure depends on where in the brain the seizure happens and what that part of the brain normally does.

“Focal seizure where awareness/consciousness are impaired” has replaced the term “complex partial seizure”. These seizures usually start in a small area of the temporal lobe or frontal lobe of the brain and involve other areas of the brain within the same hemisphere that affect alertness and awareness. Most subjects experience automatisms during a focal seizure with impaired consciousness.

“Percentage decrease in seizure frequency” is defined as the number of seizures at week 14 minus the number of seizures at baseline divided by the number of seizures at baseline multiplied by 100. In patients who are poor responders to existing AED any improvement in response particularly where the improvement is without side effects such as motor side effects on the central nervous system is highly desirable.

### DETAILED DESCRIPTION

### Preparation of Highly Purified CBD Extract

The following describes the production of the highly-purified (>98% w/w) cannabidiol extract of botanical origin which has a known and constant composition was used in the Examples below.

In summary the drug substance used is a liquid carbon dioxide extract of high-CBD containing chemotypes of Cannabis sativa L. which had been further purified by a solvent crystallization method to yield CBD. The crystallisation process specifically removes other cannabinoids and plant components to yield greater than 98% CBD. Although the CBD is highly purified because it is produced from a Cannabis plant rather than synthetically there is a small amount of other cannabinoids which are co-produced and co-extracted with the CBD. Details of these cannabinoids and the quantities in which they are present in the medication are as follows:

| Cannabinoid | Concentration |
|---|---|
| CBDV | 0.2-0.8% (w/w) |
| CBD-C4 | 0.3-0.4% (w/w) |
| CBD-C1 | 0.1-0.15% (w/w) |
| Δ⁹ THC | 0.02-0.1% (w/w) |

### Production of the Drug Product

The drug product is presented as an oral solution. The oral solution presentation contains 25 mg/ml or 100 mg/ml CBD, with the excipients sesame oil, ethanol, sucralose and flavouring. Two product strengths are available to allow dose titration across a wide dose range.

The 25 mg/ml solution is appropriate at lower doses and the 100 mg/ml solution at higher doses.

The drug product formulation is as described below:

| | Qualitative | | Reference to |
|---|---|---|---|
| Component | Composition | Function | Quality Standard |
| Cannabidiol (CBD) | 25 mg/ml or | Active | In-house |
| | 100 mg/ml | | |
| Anhydrous ethanol | 79.0 mg/ml | Excipient | Ph. Eur. |
| Sucralose | 0.5 mg/ml | Sweetener | In-house |
| Strawberry | 0.2 mg/ml | Flavouring | In-house |
| flavouring | | | |
| Sesame oil | q.s to 1.0 ml | Excipient | Ph. Eur. |

The drug substance, CBD is insoluble in water. Sesame oil was selected as an excipient to solubilize the drug substance.

A sweetener and fruit flavouring are required to improve palatability of the sesame oil solution.

Ethanol was required to solubilize the sweetener and the flavouring.

The composition can be substantially equivalent, by which is meant the functional ingredients can vary from the qualitative composition specified above by an amount of up to 10%.

Example 1 below describes the use of a highly purified Cannabis extract comprising cannabidiol (CBD). Cannabidiol is the most abundant non-psychoactive cannabinoid in the selected chemovar. Previous studies in animals have demonstrated that CBD has anticonvulsant efficacy in multiple species and models.

Example 1 describes a case study of a child with a KCNT1 mutation that was provided highly purified cannabidiol as part of an expanded access treatment program of children with refractory epilepsy.

### Example 1: Efficacy of Cannabidiol in Reducing Seizure Frequency, Seizure Severity and Other Symptoms in Children and Young Adults with Epilepsy Associated with KCNT1 Mutation

### Materials and Methods

The child was aged three years and 11 months when he was enrolled in an expanded access compassionate use program for CBD. The patient was diagnosed with Epilepsy of Infancy with Migrating Focal Seizures (EIMFS). The diagnosis by clinical and EEG criteria was corroborated by genetic testing which showed a mutation in the KCNT1 gene.

This subject was treated with a highly purified extract of cannabidiol (CBD) obtained from a Cannabis plant. Seizure frequency and severity was documented in seizure diaries at baseline (4-week pre-treatment period) and over the treatment period. At each visit quality of life changes, including mood, behaviour, and cognitive function were recorded in addition to global impression of change using a numerical scale.

The patient first presented with seizures on the first day of life. He experienced on average 14 seizures per day. The types of seizure that occurred were motor arrest; asymmetric tonic; clonic extremity movements, irregular breathing and cyanosis. The patient also had a profound developmental delay with a Developmental Quotient (DQ) score of less than 25.

The patient had tried and failed seven different anti-epileptic drugs and at the time of treatment and continued on the ketogenic diet however his seizures remained refractory.

Pharmaceutical-grade plant-derived CBD oral solution was gradually titrated by 2 to 5 mg/kg increments up to a maximum dose of 25 mg/kg/day.

The patient was seen at regular intervals of 2-4 weeks. Laboratory testing for hematologic, liver, kidney function and concomitant AED levels was performed at baseline, and after every 4 weeks of CBD therapy.

The patient was treated for over three years and remains on treatment currently.

### Results

Over the initial 12 weeks of the treatment period the overall seizure frequency increased by 13% compared to baseline. The patient was unable to reach the maximum dose of 25 mg/kg/day over the initial 12 week period due to experiencing somnolence when the CBD was titrated too quickly.

After 16 weeks of treatment the patient was able to reach a dose of 25 mg/kg/day without side effects and at this dose experienced a 12% reduction in overall seizure frequency.

The patient also experienced a clinically meaningful reduction in seizure severity. There was a 93% reduction in Type C seizures which were characterised by irregular breathing, cyanosis and clonic extremity movements. There was also a 48% reduction in Type D seizures which are the most severe type of seizures and are characterised by asymmetric tonic seizures.

In addition to the reduction in seizure frequency and severity the caregivers of the patient documented that treatment with the highly purified extract of CBD made a noticeable difference to his demeanour. After 6 months of treatment, he was described as more interactive and had attained new milestones including babbling, smiling, and fixing.

### CONCLUSIONS

These data indicate that CBD is effective in the treatment of epilepsy associated with KCNT1 mutations.

It is surprising that in this very intractable patient there was a reduction in both the frequency and the severity of seizures which has been accompanied by an improvement in developmental milestones.

### REFERENCES

Ames F R and Cridland S (1986). “Anticonvulsant effects of cannabidiol.” S Afr Med J 69:14. Consroe P, Martin P, Eisenstein D. (1977). “Anticonvulsant drug antagonism of delta-9-tetrahydrocannabinol induced seizures in rabbits.” Res Commun Chem Pathol Pharmacol. 16:1-13
Consroe P, Benedicto M A, Leite J R, Carlini E A, Mechoulam R. (1982). “Effects of cannabidiol on behavioural seizures caused by convulsant drugs or current in mice.” Eur J Pharmaco. 83:293-8
Cunha J M, Carlini E A, Pereira A E, Ramos O L, Pimental C, Gagliardi R et al. (1980). “Chronic administration of cannabidiol to healthy volunteers and epileptic patient.” Pharmacology. 21:175-85
Dravet C. The core Dravet syndrome phenotype. Epilepsia. 2011 April; 52 Suppl 2:3-9.
Eadie, M J (December 2012). “Shortcomings in the current treatment of epilepsy.” Expert Review of Neurotherapeutics 12 (12): 1419-27.
Kwan P, Arzimanoglou A, Berg A T, Brodie M J, Hauser W A, Mathern G, Moshé S L, Perucca E, Wiebe S, French J. (2009) “Definition of drug resistant epilepsy: Consensus proposal by the ad hoc Task Force of the ILAE Commission on Therapeutic Strategies.” Epilepsia.
Mechoulam R and Carlini E A (1978). “Toward drugs derived from Cannabis.” Die naturwissenschaften 65:174-9.
Porter B E, Jacobson C (December 2013). “Report of a parent survey of cannabidiol-enriched Cannabis use in paediatric treatment resistant epilepsy” Epilepsy Behaviour. 29(3) 574-7
Press C A, Knupp K G and Chapman K E (2015) “Parental reporting of response to oral Cannabis extracts for treatment of refractory epilepsy”. Epilepsy and Behaviour. 45. 49-52.
Thurman, D J; Beghi, E; Begley, C E; Berg, A T; Buchhalter, J R; Ding, D; Hesdorffer, D C; Hauser, W A; Kazis, L; Kobau, R; Kroner, B; Labiner, D; Liow, K; Logroscino, G; Medina, M T; Newton, C R; Parko, K; Paschal, A; Preux, PM; Sander, J W; Selassie, A; Theodore, W; Tomson, T; Wiebe, S; ILAE Commission on, Epidemiology (September 2011). “Standards for epidemiologic studies and surveillance of epilepsy.” Epilepsia. 52 Suppl 7: 2-26

1. A method of treating epilepsy associated with a KCNT1 mutation comprising administering cannabidiol (CBD).

2. The method of claim 1, wherein treating treats non-seizure symptoms in epilepsy associated with KCNT1 mutation.

3. The method of claim 1, wherein the epilepsy associated with KCNT1 mutation is Epilepsy of Infancy with Migrating Focal Seizures (EIMFS).

4. The method of claim 1, wherein the epilepsy is a treatment resistant epilepsy (TRE).

5. The method of claim 1, comprising administering one or more concomitant anti-epileptic drugs (AED).

6. The method of claim 1, wherein the CBD is present as a highly purified extract of Cannabis which comprises at least 98% (w/w) CBD.

7. The method of claim 6, wherein the extract comprises up to 0.1% (w/w) THC.

8. The method of claim 7, wherein the THC is present at a concentration of between 0.02 and 0.1% (w/w).

9. The method of claim 1, wherein the extract further comprises up to 1% (w/w) CBDV.

10. The method of claim 1, wherein the CBD is present as a synthetic compound.

11. The method of claim 1, wherein the dose of CBD is between 5 and 50 mg/kg/day.

12. (canceled)
